(19) United States
(12) Patent Application Publication
(10) Pub. No.: US 2009/0036983 A1
(43) Pub. Date: Feb. 5, 2009

(54) Voice Prosthesis Gel Cap Loading Tool

(75) Inventor: An Quoc Tran, Alameda, CA (US)

Correspondence Address: FREUDENBERG-NOK GENERAL PARTNERSHIP;LEGAL DEPARTMENT, 47690 EAST ANCHOR COURT, PLYMOUTH, MI, 48170-2455, US

(73) Assignee: HELIX MEDICAL, LLC, Plymouth, MI (US)

(21) Appl. No.: 12/052,397

(22) Filed: Mar. 20, 2008

Related U.S. Application Data
(63) Continuation-in-part of application No. 11/832,167, filed on Aug. 1, 2007.

(51) Int. Cl. A61F 2/20; A61F 11/00

(52) U.S. Cl. 623/9; 606/108

(57) ABSTRACT

A medical device, gel-cap loading tool. The loading tool facilitates the insertion of a component of a medical device, such as the esophageal flange of a voice prosthesis, within a dissolvable gel cap. The medical device is disposed in a cavity with the component collapsed therein. A gel cap is disposed in the cavity and is axially aligned with the medical device. An engaging member engages with at least one of the gel cap and the medical device to cause relative movement between the gel cap and the medical device which causes the component to be inserted into the gel cap.

### CROSS-REFERENCE TO RELATED APPLICATIONS

This application is a continuation-in-part of U.S. patent application Ser. No. 11/832,167 filed on Aug. 1, 2007. The disclosure of the above application is incorporated herein by reference.

### FIELD

The present disclosure relates to loading tools and, more particularly, to an apparatus for loading a gel cap over a component of a medical device, such as a flange of a voice prosthesis, wherein a reduction in radial profile is used to fit the medical device into an orifice.

### BACKGROUND AND SUMMARY

The statements in this section merely provide background and summary information related to the present disclosure and may not constitute prior art.

Various medical devices can have a radial profile that is temporarily reduced to fit the medical device into an orifice. Once such medical device is a voice prosthesis. A voice prosthesis is a one-way valve that can be placed into an orifice (puncture) between the trachea and esophagus. The voice prosthesis allows air to flow from the lungs into the esophagus to enable speech. Flanges at both ends of the voice prosthesis retain the prosthesis in the tracheoesophageal puncture.

The insertion of the esophageal flange of the voice prosthesis through the tracheoesophageal puncture can be a traumatic experience. To reduce the traumatic experience, the esophageal flange can be placed within a dissolvable capsule (referred to herein as “gel cap”, “cap” and “capsule”) to reduce the overall dimensions and facilitate the insertion through the tracheoesophageal puncture. Once in place, water or other liquid can be swallowed to dissolve the capsule and allow the esophageal flange to expand to its normal dimension and secure the voice prosthesis within the tracheoesophageal puncture.

Existing devices to load the esophageal flange within the cap may be difficult to use. New and existing users often struggle to properly load the esophageal flange into the cap. The existing devices can have a long learning curve. Additionally, the user typically wears protective gloves when handling the voice prosthesis and, therefore, limits the tactile sensation.

The present disclosure provides a loading tool. The loading tool facilitates the insertion of a component of a medical device, such as the esophageal flange of a voice prosthesis, within a dissolvable gel cap. The gel cap is disposed in a cavity with the component collapsed therein. A gel cap disposed in the cavity can be axially aligned with the medical device. An engaging member engages with at least one of the gel cap and the medical device to cause relative movement between the gel cap and the medical device which causes the component to be inserted into the gel cap.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of the various components of a loading tool according to the present disclosure in an unassembled state along with a voice prosthesis and gel cap;

FIG. 2 is a perspective view of the loading tool of FIG. 1 with the sleeve disposed in the barrel;

FIG. 3 is a perspective view of the loading tool of FIG. 2 with a voice prosthesis inserted into the loading tool;

FIG. 4 is a perspective view of the loading tool of FIG. 3 with a gel cap inserted into the loading tool;

FIG. 5 is a perspective view of the loading tool of FIG. 4 with the inserter stick engaged with the loading tool and the voice prosthesis;

FIGS. 6-7 are perspective views of the loading tool of FIG. 5 in various stages of loading the gel cap over the esophageal flange;

FIG. 8 is a perspective view of the loading tool of FIG. 7 with the gel cap loaded over the esophageal flange and the voice prosthesis coupled to the inserter stick;

FIG. 9 is a perspective view of a voice prosthesis with a gel cap over the esophageal flange;

FIG. 10 is a partial cutaway view of the tracheoesophageal puncture between the trachea and the esophagus with the gel cap dissolved and the esophageal and tracheoesophageal flanges maintaining the voice prosthesis therein;

FIG. 11 is an end view of the sleeve of the loading tool of FIG. 1;

FIG. 12 is a perspective view of an alternate configuration of the loading tool of FIG. 1 according to the present disclosure;

FIG. 13 is an assembled perspective view of the loading tool of FIG. 12 with a voice prosthesis, gel cap, and inserter stick engaged with the loading tool;

FIG. 14-15 are perspective views of the loading tool of FIG. 13 in various stages of loading the gel cap over the esophageal flange;

FIG. 16 is an alternate configuration for a sleeve that can be used with the loading tool of FIG. 1;

FIG. 17 is a perspective view of another loading tool according to the present disclosure in an unassembled state along with a voice prosthesis and gel cap;

FIG. 18 is a perspective view of the loading tool of FIG. 17 in a partially assembled state;

FIG. 19 is a perspective view of the loading tool of FIG. 18 in another partially assembled state;

FIGS. 20-22 are perspective views of the loading tool of FIG. 19 in various stages of loading the gel cap over the esophageal flange;

FIG. 23 is a perspective view of the loading tool of FIG. 22 with the gel cap on the esophageal flange and the voice prosthesis coupled to the inserter stick and ready to be inserted into the tracheoesophageal puncture;

FIG. 24 is a perspective view of another loading tool according to the present disclosure in an unassembled state along with a voice prosthesis and gel cap;

FIG. 25 is a cross-sectional view of the loading tool of FIG. 24 along line 25-25 with the voice prosthesis and gel cap loaded therein;

FIG. 26 is a cross-sectional view of the loading tool of FIG. 25 illustrating the loading of the esophageal flange into the gel cap;

FIG. 27 is a cross-sectional view of the loading tool of FIG. 26 showing the removal of the voice prosthesis and gel cap therefrom;

FIG. 28 is a perspective view of yet another loading tool according to the present disclosure in an unassembled state along with a voice prosthesis and gel cap;

FIG. 29 is a cross-sectional view of the loading tool of FIG. 28 along line 29-29 with the voice prosthesis and gel cap loaded therein;

FIG. 30 is a cross-sectional view of the loading tool of FIG. 29 illustrating the loading of the esophageal flange into the gel cap;

FIG. 31 is a perspective view of the loading tool of FIG. 30 showing the removal of the voice prosthesis and gel cap therefrom;

FIG. 32 is a perspective view of still another loading tool according to the present disclosure in an unassembled state along with a voice prosthesis and gel gap;

FIG. 33 is a cross-sectional view of the loading tool of FIG. 32 along line 33-33 with the voice prosthesis and gel cap loaded therein;

FIG. 34 is a cross-sectional view of the loading tool of FIG. 33 illustrating the loading of the gel cap over the esophageal flange; and

FIG. 35 is a perspective view of the loading tool of FIG. 34 showing the removal of the voice prosthesis and gel cap therefrom.

### DETAILED DESCRIPTION

The following description is merely exemplary in nature and is not intended to limit the present disclosure, application, or uses.

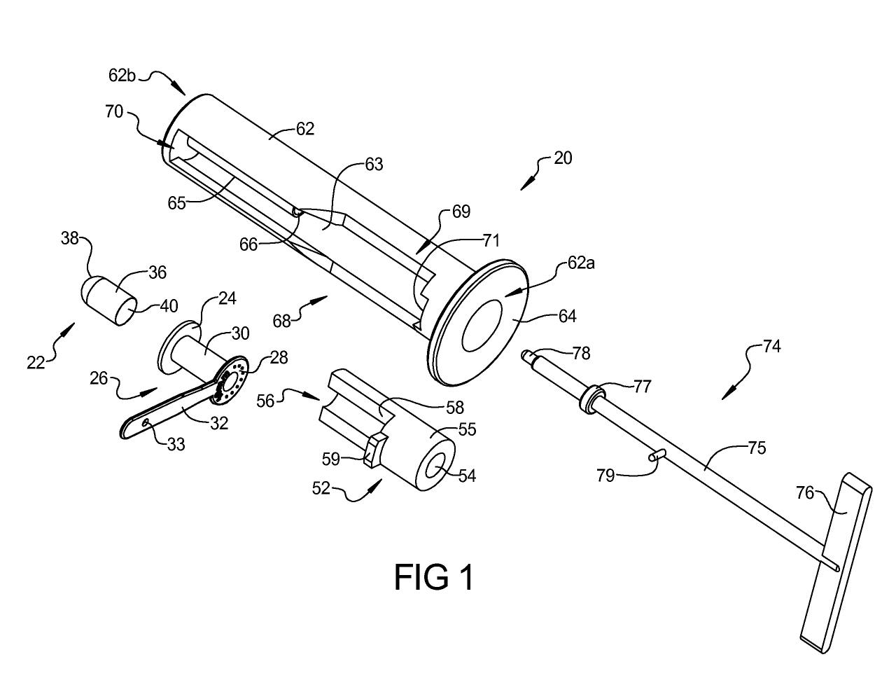

Referring to FIG. 1, a gel cap loading tool 20, according to the present disclosure, is shown. Loading tool 20 is operable to place a gel cap 22 over a component of a medical device having a radial profile that is to be reduced in dimension and inserted into gel cap 22, such as an esophageal flange 24 of a voice prosthesis 26, by way of non-limiting example. Voice prosthesis 26 includes esophageal flange 24 and a tracheal flange 28 with a generally cylindrical hollow body 30 extending there between. Body 30 is open on both flanges 24, 28. A one-way valve (not shown) is disposed within body 30. A strap 32 extends outwardly from tracheal flange 28. Strap 32 has an opening 33 adjacent its end. The depiction of voice prosthesis 26 is representative of typical voice prosthesis. It should be appreciated, however, that voice prosthesis 26 can have a different configuration than that shown and still be utilized with the loading tool 20 according to the present disclosure.

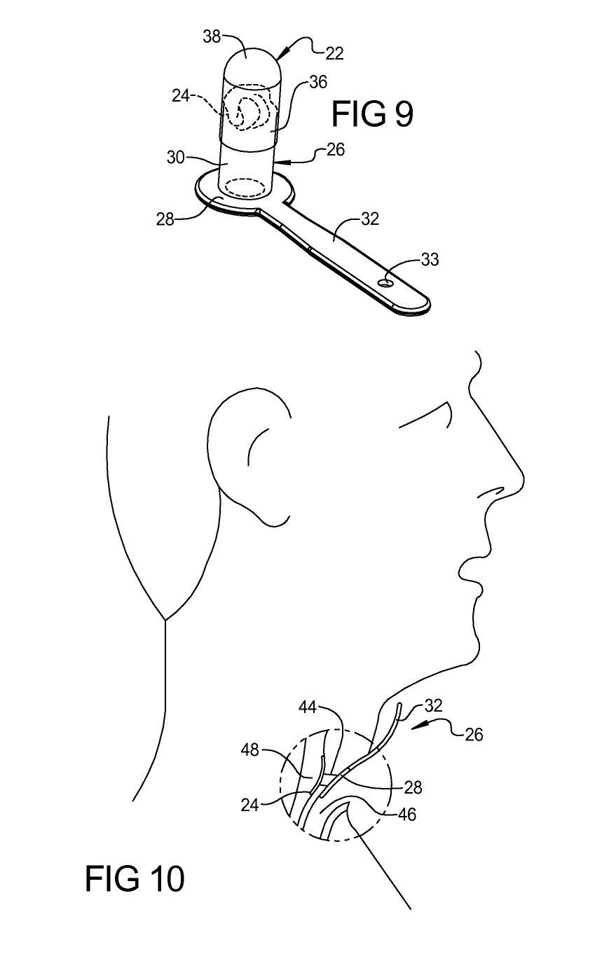

Gel cap 22 includes a generally cylindrical body portion 36 and a hemispherical end portion 38. Body portion 36 and end portion 38 define an interior cavity 40 within which esophageal flange 24 and a portion of body 30 of voice prosthesis 26 can be inserted, as shown in FIG. 9. Gel cap 22 is dissolvable and facilitates the insertion of voice prosthesis 26 into a tracheoesophageal puncture 44 that extends between a trachea 46 and esophagus 48 of a user, as shown in FIG. 10. In FIG. 10, gel cap 22 has been dissolved such that esophageal flange 24 is fully expanded within esophagus 48. Esophageal and tracheal flanges 24, 28 thereby retain voice prosthesis 26 in the tracheoesophageal puncture 44, as known in the art.

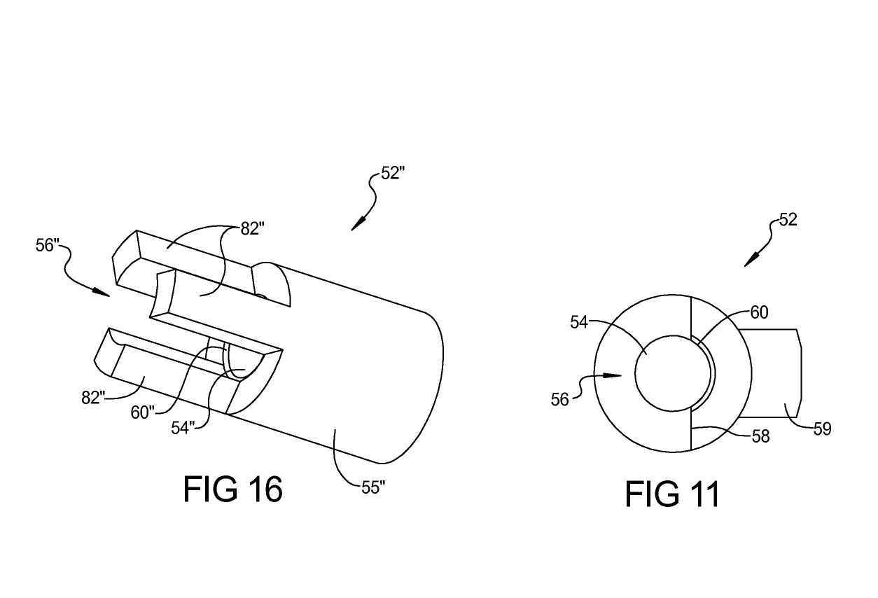

Referring to FIGS. 1 and 11, loading tool 20 includes a generally cylindrical sleeve 52 with a cylindrical opening/cavity 54 extending longitudinally therethrough. A portion of a cylindrical periphery 55 of sleeve 52 is missing to provide a loading opening 56. As shown in FIG. 11, loading opening 56 extends less than 180 degrees about the cylindrical periphery 55 and provides a substantially flat surface 58 which is off-center of a longitudinal axis of sleeve 52. Loading opening 56 facilitates the insertion of voice prosthesis 26 into sleeve 52, as described below. A tab 59 extends radially outwardly from an end of loading opening 56. The access to opening 54 through loading opening 56 can include a chamfer 60 to facilitate the insertion of voice prosthesis 26 into opening 54. When inserted into opening 54, the diameter of opening 54 dictates the compression/collapsing of esophageal flange 24 therein. The diameter of opening 54 is chosen so that esophageal flange 24 collapses a sufficient amount to fit within interior 40 of gel cap 22 without causing undue stress on esophageal flange 24 and gel cap 22 during the loading process.

Loading tool 20 includes an elongated generally cylindrical barrel 62 with opposite ends 62a, 62b and with a cylindrical interior cavity 63 that extends longitudinally therebetween. End 62a is open to cavity 63 and includes a flange 64 that extends radially outwardly therefrom. Barrel 62 includes a generally cylindrical stop 65 that extends longitudinally from closed end 62b toward open end 62a. The outer diameter of stop 65 is smaller than the inner diameter of cavity 63 such that stop 65 is completely spaced apart from the inner surface of cavity 63. Stop 65 can be coaxial with barrel 62 and cavity 63. Stop 65 includes a concave recess 66 on the free end thereof. Stop 65 is fixed in position within barrel 62. The outer diameter of stop 65 is smaller than the inner diameter of opening 54 in sleeve 52 to allow sleeve 52 to slide over stop 65.

Barrel 62 includes a longitudinally extending access opening 68 that extends from the closed end 62b of barrel 62 toward flange 64. Opening 68 includes a wide portion 69 and a narrow portion 70. Wide portion 69 is dimensioned to allow sleeve 52 to be inserted into cavity 63 of barrel 62. Wide portion 69 includes a recess 71 that is configured to receive tab 59 of sleeve 52. Narrow portion 70 is dimensioned to allow tab 59 to slide therethrough when sleeve 52 is slid within cavity 63.

An implement, such as an inserter stick 74, can be used to insert voice prosthesis 26 within tracheoesophageal puncture 44. Inserter stick 74 includes a stem 75 with a handle 76 at one end thereof. There is a radial collar 77 on stem 75 adjacent the opposite end. Collar 77 limits the distance inserter stick 74 can be inserted into voice prosthesis 26. A tip 78 on the end of stem 75 can be tapered to facilitate insertion of stem 75 into voice prosthesis 26 through the opening in tracheal flange 28. A peg 79 extends outwardly from stem 75 between collar 77 and handle 76. Peg 79 is configured to receive strap 32 with peg 79 extending through opening 33. Peg 79 is positioned so that strap 32 is stretched to engage with peg 79. The stretching of strap 32 helps keep voice prosthesis 26 on inserter stick 74 during handling.

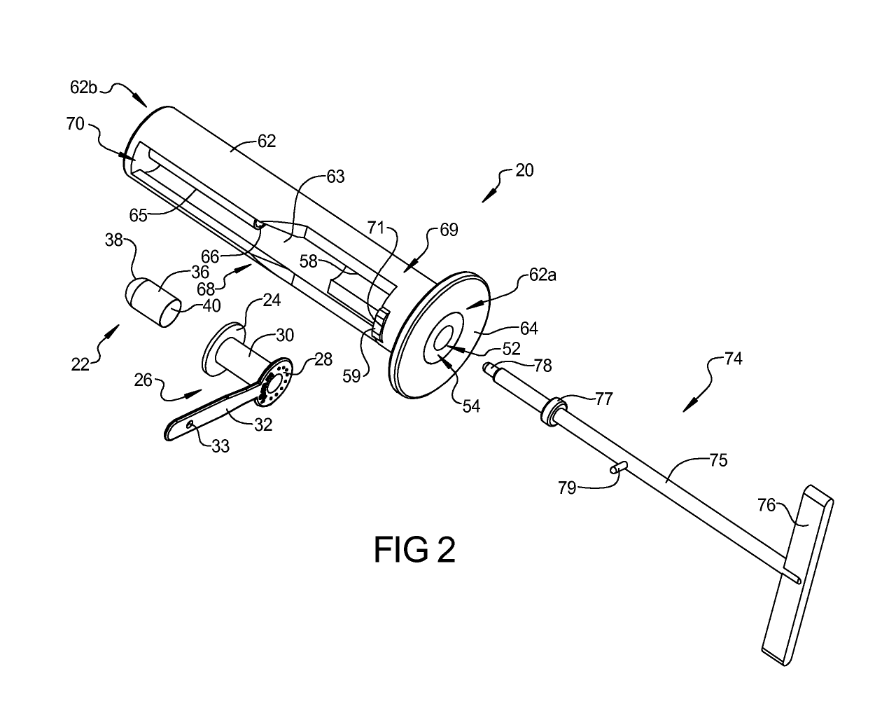

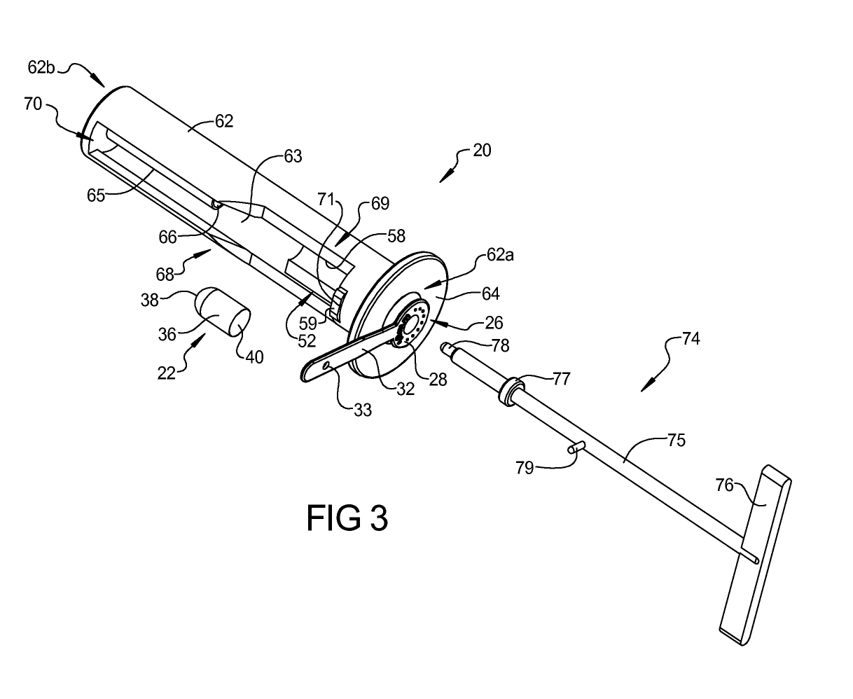

Referring now to FIGS. 2-8, the operation of loading tool 20 to insert esophageal flange 24 of voice prosthesis 26 within gel cap 22 is shown. Initially, as shown in FIG. 2, sleeve 52 is disposed within cavity 63 of barrel 62. Sleeve 52 is positioned adjacent flange 64 with tab 59 engaged in recess 71. Engagement of tab 59 with recess 71 inhibits rotation of sleeve 52 within barrel 62. Next, voice prosthesis 26 is inserted into opening 54 of sleeve 52 by threading strap 32 through loading opening 56 and out opening 54 and end 62a of barrel 62. Strap is threaded so that it extends beyond flange 64 of barrel 62. Strap 32 can then be pulled while the remaining portions of voice prosthesis 26 are inserted into opening 54 through loading opening 56. During the loading process, the flanges 28, 24 will be compressed and collapsed within opening 54, thereby reducing their radial dimensions. When completely seated within sleeve 52, tracheal flange 28 may extend out of opening 54 along the end of sleeve 52 while esophageal flange 24 remains compressed such that its radial dimension is reduced, as shown in FIG. 3. In some embodiments, tracheal flange 28 may remain compressed in opening 54 with its radial dimension reduced along with esophageal flange 24.

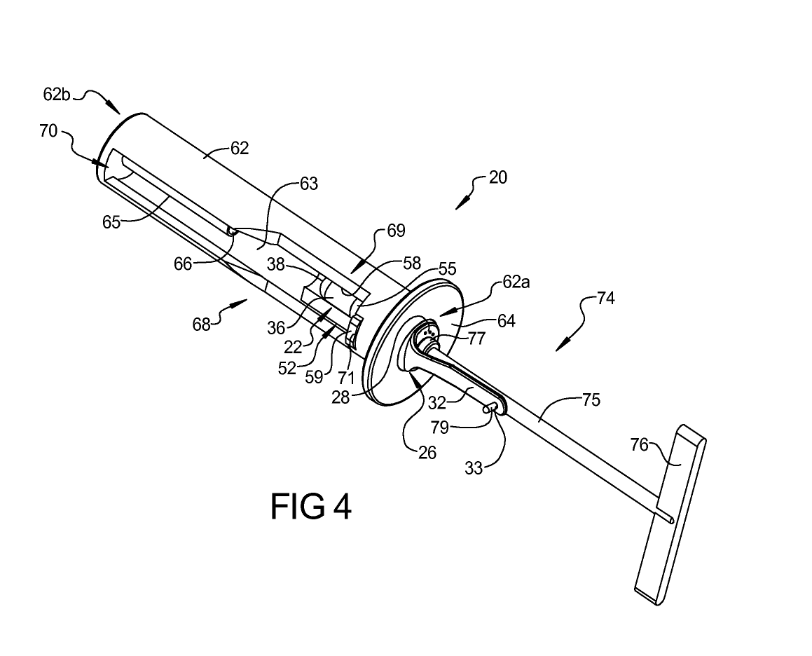

The next step is to load gel cap 22 into sleeve 52. Gel cap 22 is inserted into loading opening 56 of sleeve 52. Because loading opening 56 extends less than 180 degrees around the cylindrical periphery 55, gel cap 22 can be snapped into loading opening 56, as shown in FIG. 4. Opening 54 of sleeve 52 axially aligns voice prosthesis 26 and gel cap 22. Next, as shown in FIG. 5, tip 78 and stem 75 on inserter stick 74 are inserted into voice prosthesis 26 through the opening in tracheal flange 28. Strap 32 is then stretched and secured to inserter stick 74 by engaging peg 79 with opening 33.

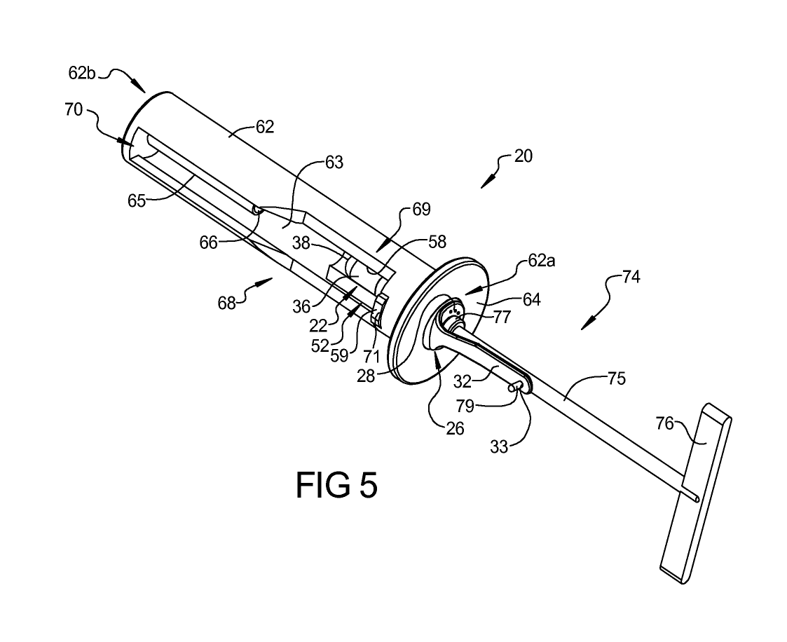

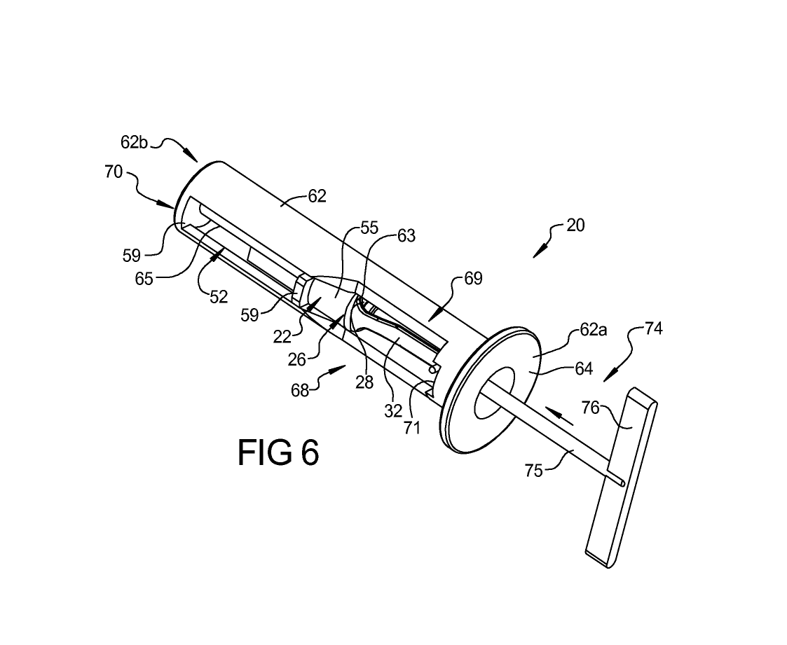

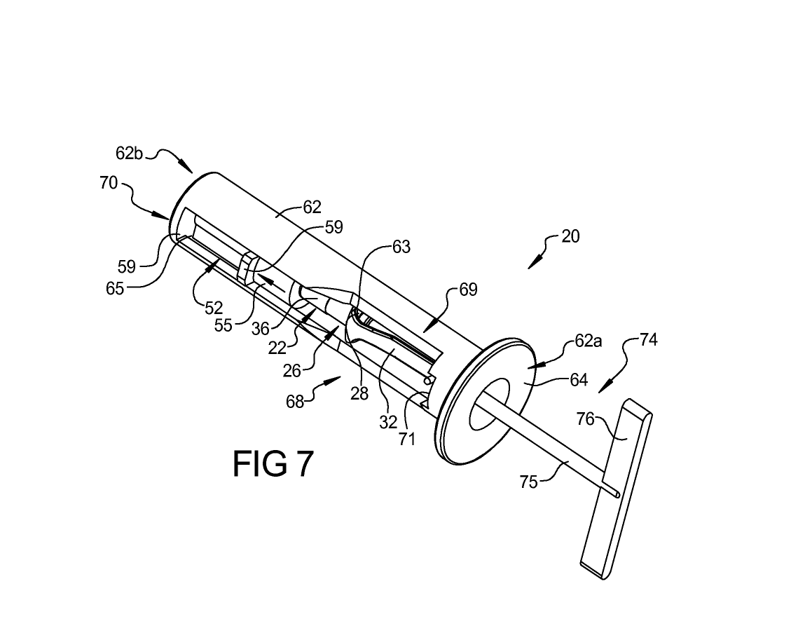

Next, as shown in FIG. 6, sleeve 52 and inserter stick 74 are moved axially relative to barrel 62 so that sleeve 52 approaches stop 65. Flange 64 and handle 76 can be moved toward one another like a syringe to facilitate the movement of inserter stick 74 and sleeve 52 relative to barrel 62. The movement of inserter stick 74 and sleeve 52 relative to barrel 62 also causes voice prosthesis 26 to move with the movement of sleeve 52 and inserter stick 74. The movement eventually causes end 38 of gel cap 22 to engage with concave recess 66 of stop 65. Stop 65 prevents additional movement of gel cap 22 relative to barrel 62. Continued movement of sleeve 52, inserter stick 74, and voice prosthesis 26 causes esophageal flange 24 to enter into interior 40 of gel cap 22. The insertion of esophageal flange 24 into interior 40 can cause some resistance to be felt by the user. Once esophageal flange 24 is completely disposed within gel cap 22, continued movement of inserter stick 74 and voice prosthesis 26 relative to barrel 62 stops. At this time, the user can then continue to move sleeve 52 relative to barrel 62 by pushing tab 59 toward closed end 62b of barrel 62. Sleeve 52 then moves relative to voice prosthesis 26 and gel cap 22 such that sleeve 52 disengages from voice prosthesis 26 and gel cap 22, as shown in FIG. 7.

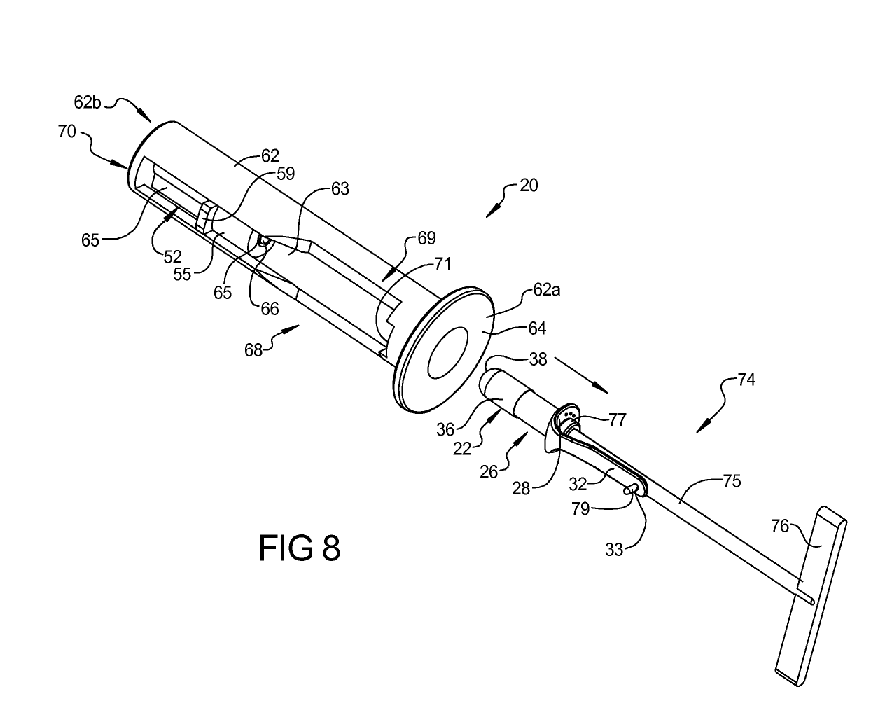

Next, inserter stick 74 is withdrawn from barrel 62. The engagement of strap 32 with peg 79 causes voice prosthesis 26 and gel cap 22 to be removed from barrel 62 along with inserter stick 74, as shown in FIG. 8. The voice prosthesis 26 can then be inserted into the tracheoesophageal puncture 44 and, once inserted, released from inserter stick 74 by disengaging strap 32 from peg 79. Inserter stick 74 can then be removed from voice prosthesis 26 and voice prosthesis 26 will remain within tracheoesophageal puncture 44. The user can then drink a fluid to dissolve gel cap 22, thereby releasing esophageal flange 24 and securing voice prosthesis 26 within tracheoesophageal puncture 44, as shown in FIG. 10.

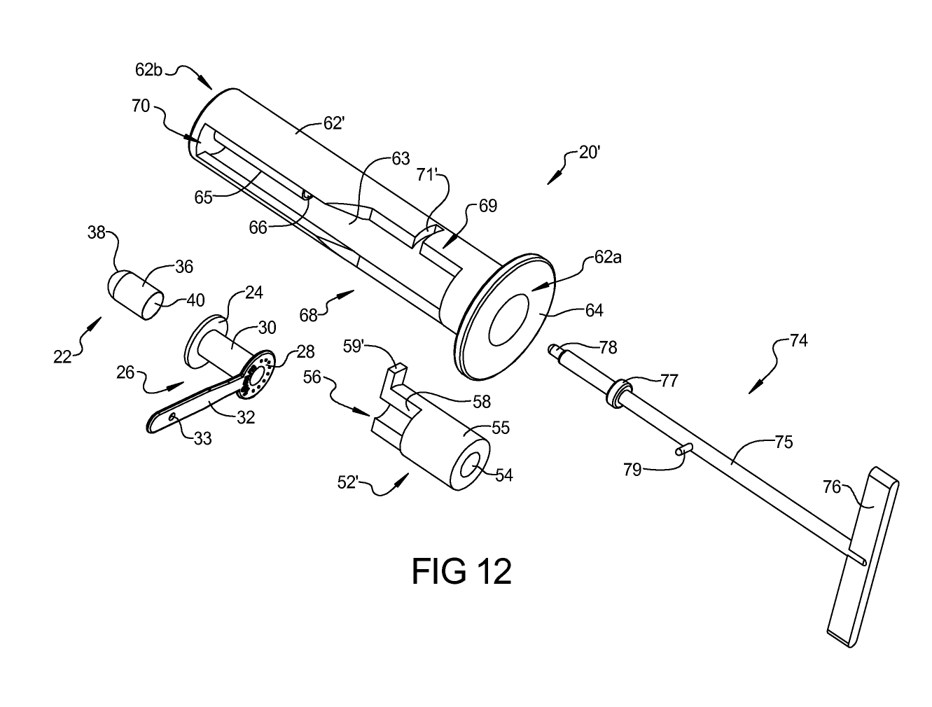

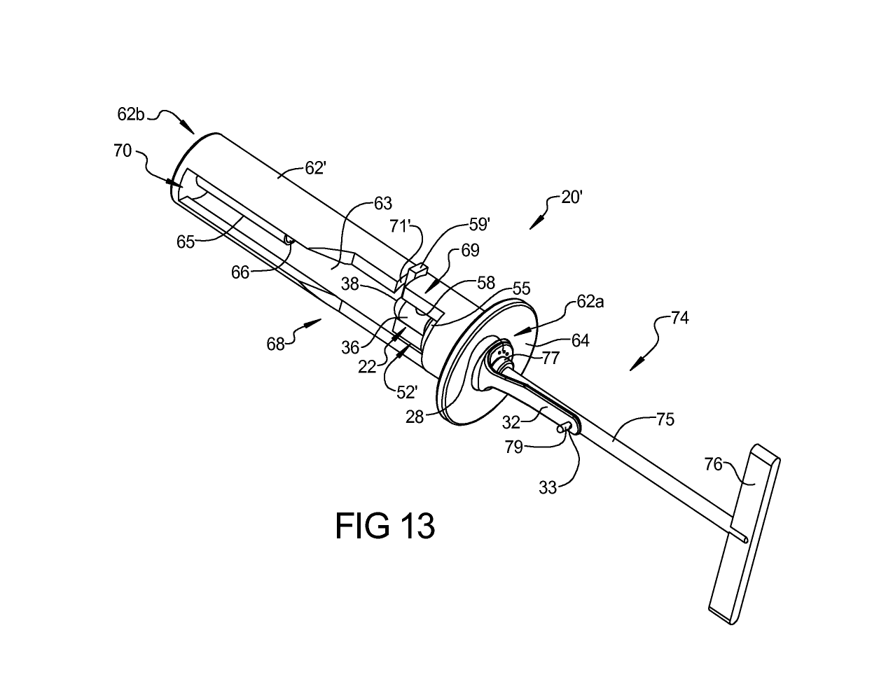

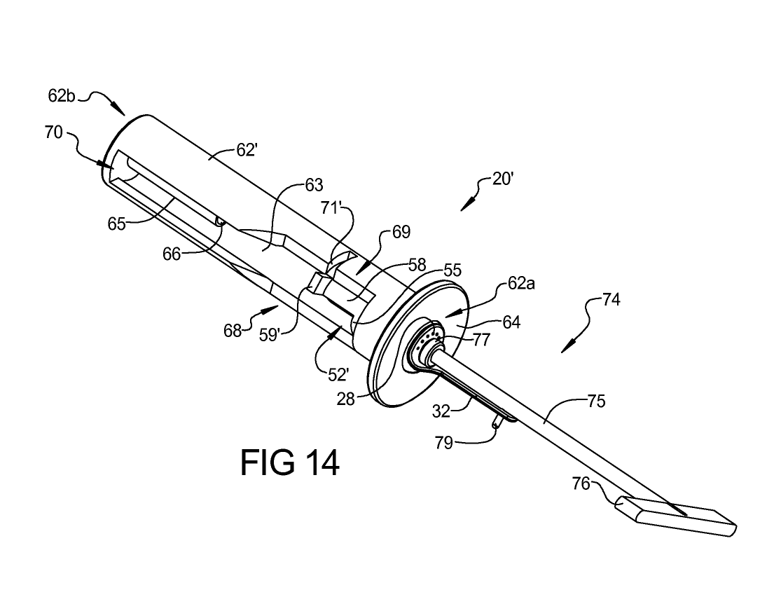

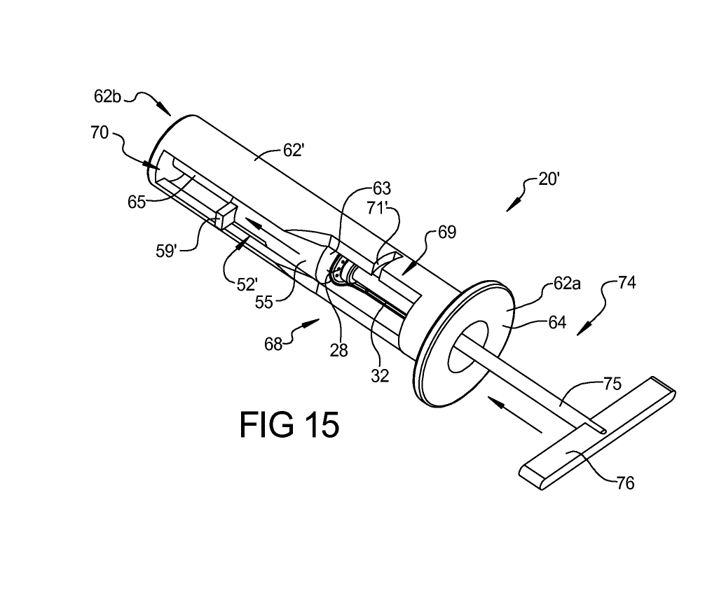

Referring now to FIGS. 12-15, an alternate configuration of a loading tool 20′ is shown. In loading tool 20′, tab 59′ of sleeve 52′ extends radially outwardly from an end of sleeve 52′ adjacent loading opening 56. Additionally, recess 71′ of barrel 62′ extends from a side of wide portion 69 and is configured to receive tab 59′ of sleeve 52′. Specifically, as shown in FIG. 13, when sleeve 52′ is inserted in barrel 62′, sleeve 52′ is rotated so that tab 59′ is engaged with recess 71′. Engagement of tab 59′ with recess 71′ inhibits longitudinal movement of sleeve 52′ within barrel 62′. When using loading tool 20′, sleeve 52′, after receiving voice prosthesis 26 and gel cap 22, is rotated within barrel 62′ such that tab 59′ is disengaged from recess 71′, as shown in FIG. 14. With tab 59′ disengaged from recess 71′, sleeve 52′ and inserter stick 74 are moved axially relative to barrel 62′ so that sleeve 52′ approaches stop 65 and causes esophageal flange 24 to enter interior 40 of gel cap 22, in the same manner described above with reference to FIGS. 1-8.

Referring now to FIG. 16, another alternate configuration of a sleeve 52″ is shown. In sleeve 52″, a plurality of longitudinally extending arms 82″ extend forwardly from cylindrical periphery 55″. Arms 82″ are spaced apart to form a loading opening 56″ that leads to opening 54″. A chamfer 60″ can surround the access to opening 54″ through loading opening 56″. In this embodiment, voice prosthesis 26 is inserted into opening 54″ by squeezing voice prosthesis 26 through the loading openings 56″ formed between arms 82″. Additionally, sleeve 52″ does not include a tab. Rather, in this embodiment, sleeve 52″ can be moved forwardly with movement of inserter stick 74 relative to barrel 62. When gel cap 22 is engaged with concave recess 66 of stop 65, further forward movement of sleeve 52″ can be performed by a user using their fingers to push sleeve 52″ forwardly such that sleeve 52″ disengages from voice prosthesis 26 and gel cap 22.

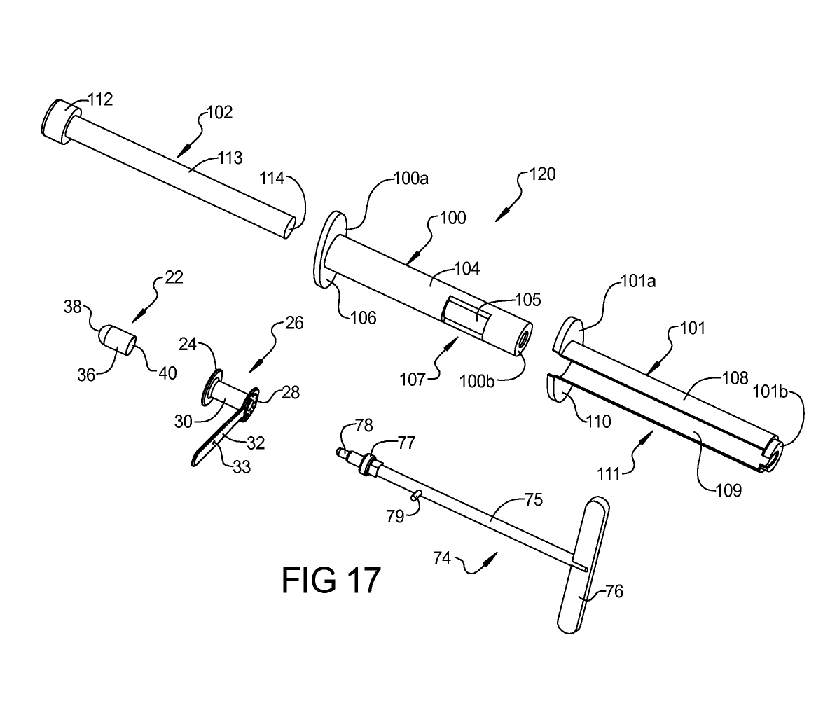

Referring now to FIGS. 17-21, another loading tool 120, according to the present disclosure, is shown. Loading tool 120 is operable to place gel cap 22 over esophageal flange 24 of voice prosthesis 26. Loading tool 120 includes inner and outer barrels 100, 101 and a plunger 102. Inner barrel 100 is generally cylindrical with an exterior peripheral surface 104 and an interior cylindrical cavity 105 that extends longitudinally throughout the entire length of inner barrel 100. Cavity 105 is accessible from both ends 100a, 100b of inner barrel 100. End 100a includes a flange 106 that extends radially outwardly therefrom. Inner barrel 100 includes an access opening 107 in peripheral surface 104. Access opening 107 allows voice prosthesis 26 and gel cap 22 to be inserted into cavity 105, as described below.

Outer barrel 101 is also cylindrical with an exterior peripheral surface 108 and an interior cylindrical cavity 109. Cavity 109 extends longitudinally within outer barrel 101 an entire length of outer barrel 101. Cavity 109 is accessible from both ends 101a, 101b of outer barrel 101. Outer barrel 101 includes a flange 110 that extends radially outwardly from end 101a. Outer barrel 101 includes an access opening 111 in peripheral surface 108 that extends an entire length of outer barrel 101 between ends 101a, 101b. Cavity 109 is dimensioned to allow inner barrel 100 to be disposed therein, as described below.

Plunger 102 includes a head 112 and an elongated cylindrical stem 113 extending therefrom. Stem 113 has an outer diameter dimensioned to fit within cavity 105 of inner barrel 100 through flange end 100a. Head 112 is larger than the opening to cavity 105 in flange end 100a and limits the travel of plunger 102 within inner barrel 100. The free end 114 of plunger 102 can include a concave recess to engage with end 38 of gel cap 22. Plunger 102 is operable to push gel cap 22 over esophageal flange 24 of voice prosthesis 26 within inner barrel 100, as described below.

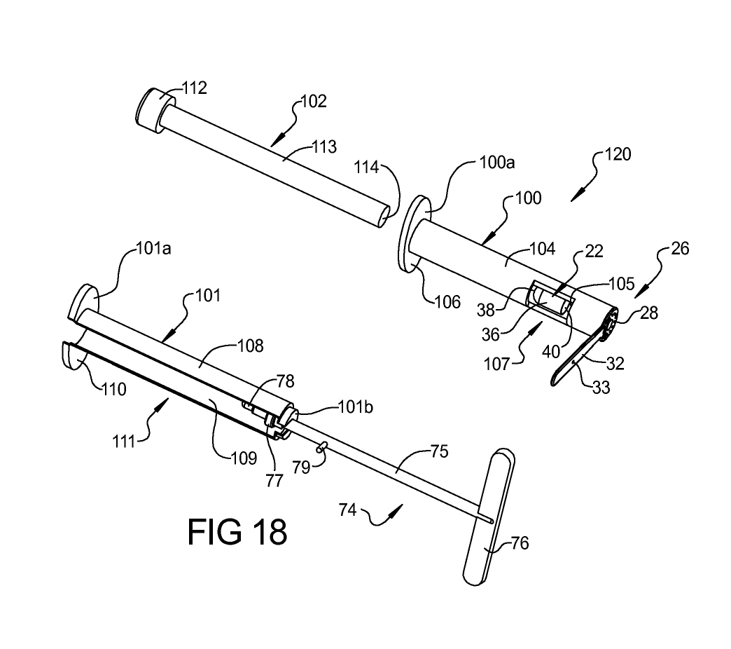

To use loading tool 120, voice prosthesis 26 can be loaded into inner barrel 100 through access opening 107. Specifically, strap 32 can be threaded through access opening 107 and out of end 100b. Strap 32 can be used to pull voice prosthesis 26 into cavity 105. When seated within inner barrel 100, tracheal flange 28 may extend out of inner barrel 100 through end 100b while esophageal flange 24 is collapsed/compressed within cavity 105, as shown in FIG. 18, such that its radial dimension is reduced. In some embodiments, tracheal flange 28 may remain compressed within cavity 105 along with esophageal flange 24. Next, gel cap 22 can be inserted into cavity 105 through access opening 107, also as shown in FIG. 18. Voice prosthesis 26 and gel cap 22 are axially aligned by cavity 105. Inserter stick 74 is inserted into cavity 109 of outer barrel 101 through access opening 111 with collar 77 disposed in cavity 109 inside of end 101b, as shown in FIG. 18.

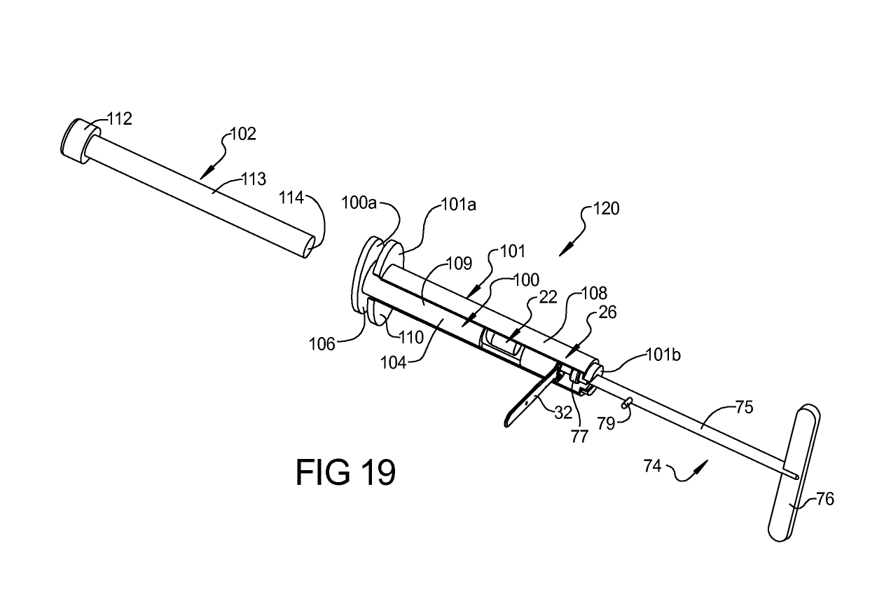

Next, inner barrel 100 is inserted into cavity 109 of outer barrel 101 through the opening in flange end 101a. Strap 32 can extend out of outer barrel 101 through access opening 111, as shown in FIG. 19. Inner barrel 100 is moved (to the right as shown in the figures) within outer barrel 101 to seat stem 75 of inserter stick 74 in voice prosthesis 26 through the opening in tracheal flange 28. Inner and outer barrels 100, 101 can be moved relative to one another in a syringe-like manner through the use of flanges 106, 110.

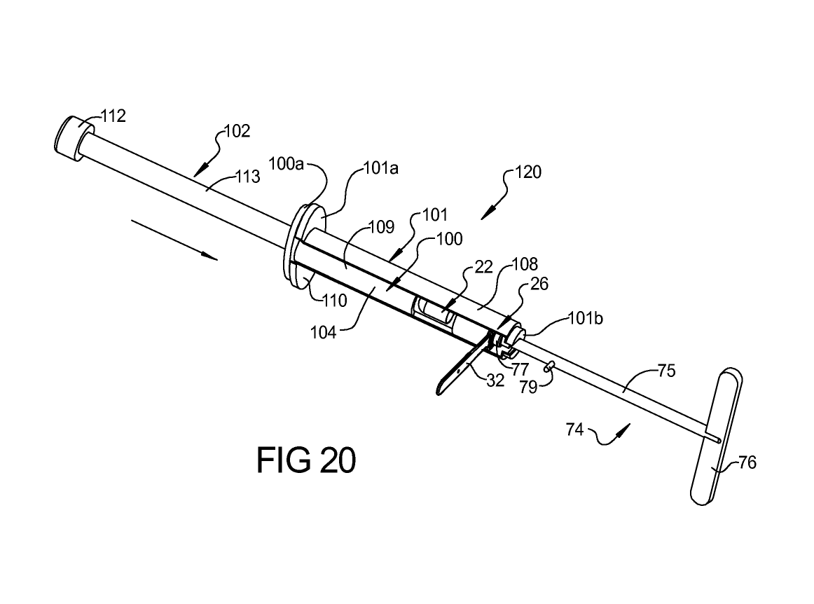

Next, as shown in FIG. 20, stem 113 of plunger 102 is inserted into cavity 105 of inner barrel 100 through the opening in flange end 100a. Loading tool 120 is now ready to be operated to push gel cap 22 over esophageal flange 24. To accomplish this, head 112 of plunger 102 is pushed toward flange end 100a of inner barrel 100. This can be accomplished using a syringe-like motion by holding onto flange 110 of outer barrel 101 while pushing on head 112 of plunger 102.

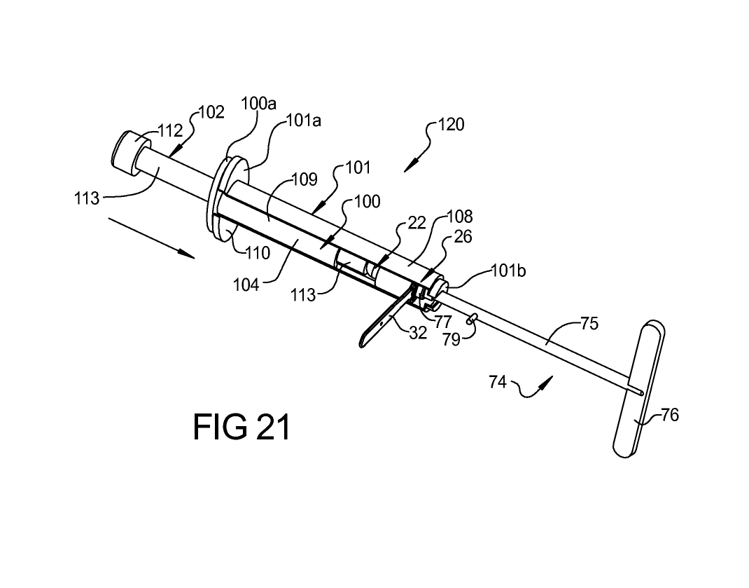

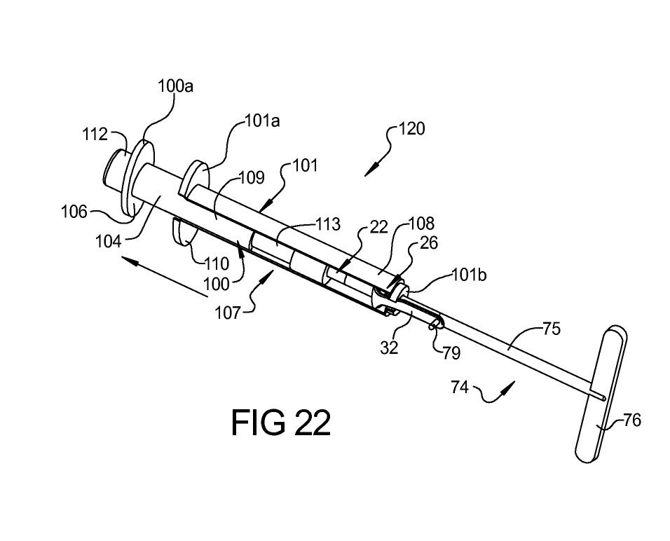

The movement of plunger 102 relative to inner barrel 100 causes stem 113 to push gel cap 22 relative to voice prosthesis 26 and onto esophageal flange 24 and a portion of body 30, as shown in FIG. 21. With gel cap 22 fully loaded on voice prosthesis 26, inner barrel 100 can be moved (to the left as shown in the figures) relative to outer barrel 101 to release voice prosthesis 26 with gel cap 22 thereon from cavity 105 of inner barrel 100 through the opening in end 100b, as shown in FIG. 22.

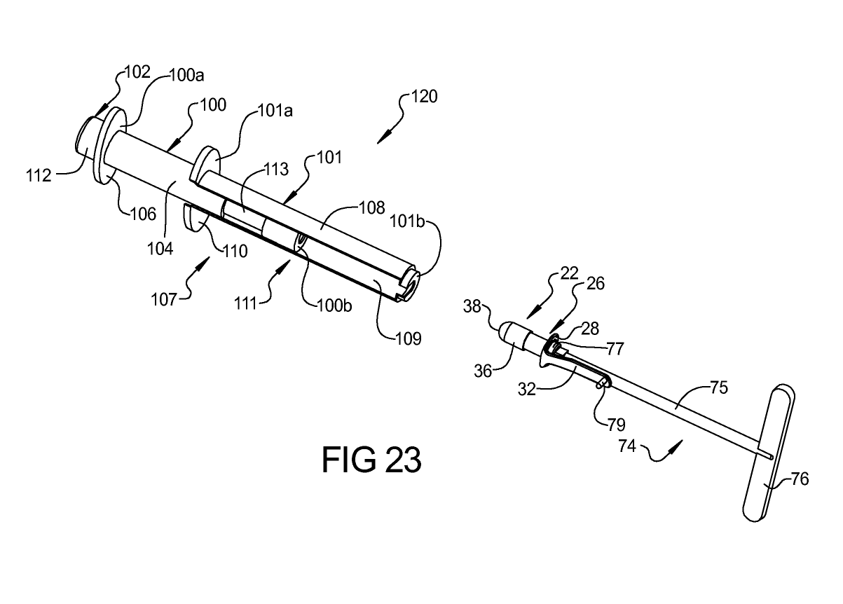

With gel cap 22 and voice prosthesis 26 being removed from cavity 105 of inner barrel 100, voice prosthesis 26 and gel cap 22 can be removed from outer barrel 101 through access opening 111, as shown in FIG. 23. Inserter stick 74 can then be utilized to place voice prosthesis 26 into the tracheoesophageal puncture 44 of a user, as shown in FIG. 10 and as described above. Thus, loading tool 120 can be utilized to position a gel cap 22 over an esophageal flange 24 of voice prosthesis 26.

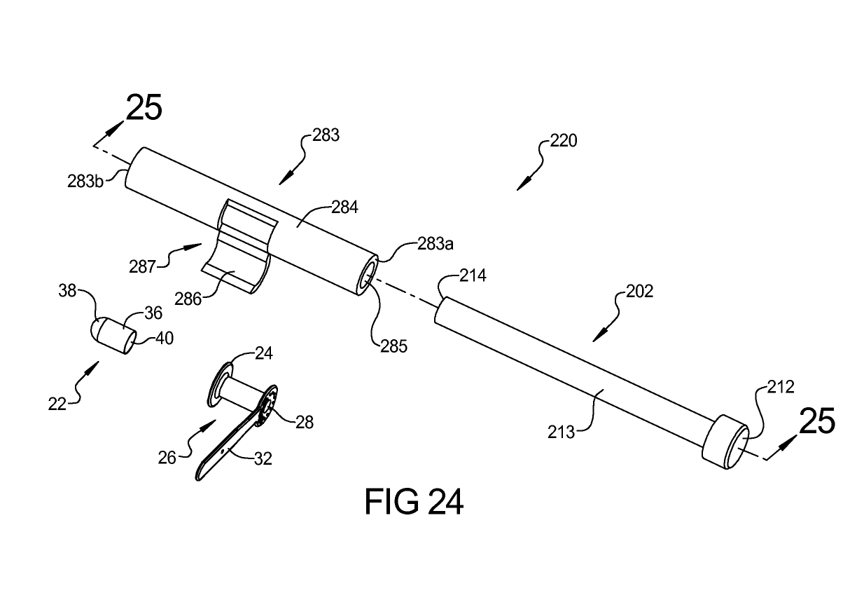

Referring now to FIGS. 24-27, another loading tool 220, according to the present disclosure, is shown. Loading tool 220 is operable to place an esophageal flange 24 of voice prosthesis 26 into a gel cap 22. Loading tool 220 includes a barrel 283 and a plunger 202. Barrel 283 is cylindrical with an exterior peripheral surface 284 and an interior cylindrical cavity 285 that extends longitudinally within barrel 283. Cavity 285 is accessible from an open end 283a of barrel 283 and is not accessible from closed end 283b of barrel 283. Barrel 283 includes an access door or hatch 286 in the exterior surface 284 that can be moved between open and closed positions to expose an access opening 287 in exterior surface 284. Hatch 286 can be pivotally coupled to barrel 283 such as through a living hinge or the like, by way of non-limiting example. The coupling of hatch 286 to barrel 283 allows hatch 286 to be manually moved between open and closed positions to respectively allow access to and close access to cavity 285 through access opening 287. When hatch 286 is open, access opening 287 is exposed and allows voice prosthesis 26 and gel cap 22 to be inserted into cavity 285, as described below. Cavity 285 includes an internal stop 288 adjacent closed end 283b. Stop 288 can be concave to generally conform to the curved end 38 of gel cap 22.

Plunger 202 includes a head 212 and an elongated cylindrical stem 213 extending therefrom. Stem 213 has an outer diameter dimensioned to fit within cavity 285 of barrel 283 through open end 283a. Head 212 is larger than the opening to cavity 285 in open end 283a and limits the travel of plunger 202 within barrel 283. The free end 214 of plunger 202 can include a generally flat surface to engage with tracheal flange 28 of voice prosthesis 26. Plunger 202 is operable to push esophageal flange 24 of voice prosthesis 26 into interior 40 of gel cap 22 within barrel 283, as described below.

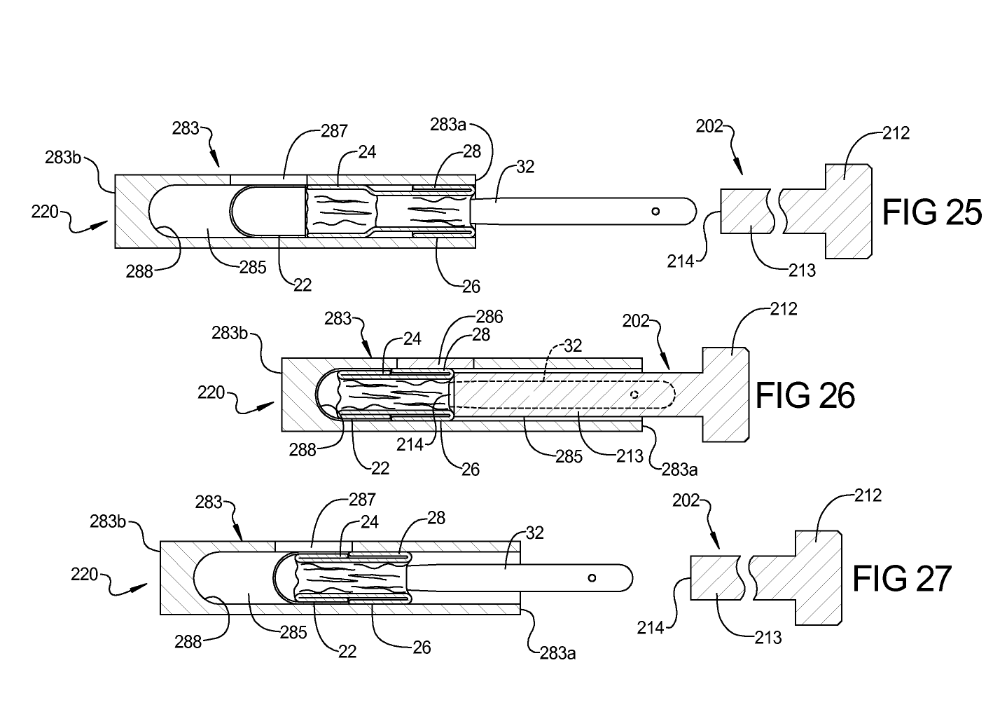

To use loading tool 220, hatch 286 is moved from the closed position to the open position, if not already open. Voice prosthesis 26 is then inserted into cavity 285 through access opening 287. Specifically, strap 32 is fed into access opening 287 toward open end 283a. Tracheal flange 28 can be manually collapsed and pushed through access opening 287 along with body 30 and esophageal flange 24, if necessary, until strap 32 extends from open end 283a of barrel 283. Strap 32 can then be pulled to facilitate the loading of voice prosthesis 26 within cavity 285. Voice prosthesis 26 is pulled within cavity 285 toward open end 283a a sufficient distance to allow gel cap 22 to also be inserted into cavity 285 through access opening 287 without interference from voice prosthesis 26. Flanges 24, 28 can collapse within cavity 285. Next, gel cap 22 is inserted into cavity 285 through access opening 287. Gel cap 22 can be positioned within cavity 285 generally aligned with access opening 287 or pushed toward stop 288 on closed end 283b. Gel cap 22 is positioned in cavity 285 with end 38 facing stop 288 and access to interior 40 facing voice prosthesis 26, as shown in FIG. 26.

Next, hatch 286 is moved from the open position to the closed position. Hatch 286 can be maintained in the closed position by the hand of the user operating loading tool 220. Next, as shown in FIG. 26, stem 213 of plunger 202 is inserted into cavity 285 through open end 283a. Loading tool 220 is now ready to be operated to push esophageal flange 24 into gel cap 22. To accomplish this, head 212 of plunger 202 is pushed toward open end 283a of barrel 283. This movement pushes voice prosthesis 26 toward gel cap 22. When gel cap 22 contacts stop 288, esophageal flange 24 will deform and enter into interior 40 of gel cap 22 with continued movement of plunger 202.

Next, plunger 202 can be removed from barrel 283. Voice prosthesis 26 with gel cap 22 thereon can then be removed from barrel 283 by pulling strap 32 from open end 283a. In this manner, voice prosthesis 26 and gel cap 22 can be removed from loading tool 220. Voice prosthesis 26 is then ready to be inserted into a trachealesophageal puncture 44 of a user, as shown in FIG. 10 and as described above.

Alternatively, in some embodiments voice prosthesis 26 with gel cap 22 disposed thereon can be removed from barrel 283 through access opening 287. When this is the case, hatch 286 is opened and voice prosthesis 26 is removed from cavity 285 through access opening 287. During this movement, gel cap 22 remains disposed over esophageal flange 24. Thus, loading tool 220 can be utilized to insert an esophageal flange 24 of a voice prosthesis 26 into interior 40 of a gel cap 22.

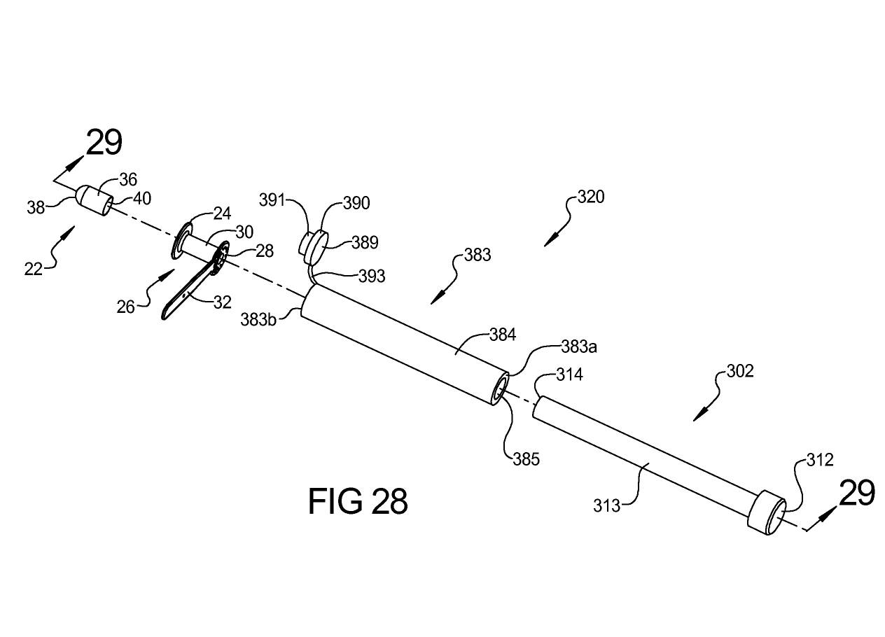

Referring now to FIGS. 28-31, another loading tool 320 according to the present disclosure is shown. Loading tool 320 is operable to insert esophageal flange 24 of voice prosthesis 26 into interior 40 of gel cap 22. Loading tool 320 includes a barrel 383 and a plunger 302. Barrel 383 is generally cylindrical with an exterior peripheral surface 384 and an interior cylindrical cavity 385 that extends longitudinally throughout the entire length of barrel 383. Cavity 385 is accessible from both ends 383a, 383b of barrel 383. A plug 389 can be attached to barrel 383 adjacent end 383b. Plug 389 includes a head portion 390 and a stem 391 extending therefrom. The free end of stem 391 can include a concave stop 392 that is contoured to be generally complementary to end 38 of gel cap 22. Stem 391 is dimensioned to fit within cavity 385. A strap 393 can be used to retain plug 389 secured to barrel 383. Barrel 383, plug 389, and strap 393 can be a single integral component. Alternatively, barrel 383 and plug 389 can be separate discrete components. The use of a strap 393 is optional and loading tool 320 can be used without the existence of strap 393. End 383b of barrel 383 is dimensioned to allow voice prosthesis 26 and gel cap 22 to be inserted into cavity 385 therethrough, as described below.

Plunger 302 includes a head 312 and an elongated cylindrical stem 313 extending therefrom. Stem 313 has an outer diameter dimensioned to fit within cavity 385 of barrel 383 through open end 383a. Head 312 is larger than the opening to cavity 385 in end 383a and limits the travel of plunger 302 within barrel 383. The free end 314 of plunger 302 can include a generally flat surface that engages with tracheal flange 28 of voice prosthesis 26. Plunger 302 is operable to push esophageal flange 24 of voice prosthesis 26 into interior 40 of gel cap 22, as described below.

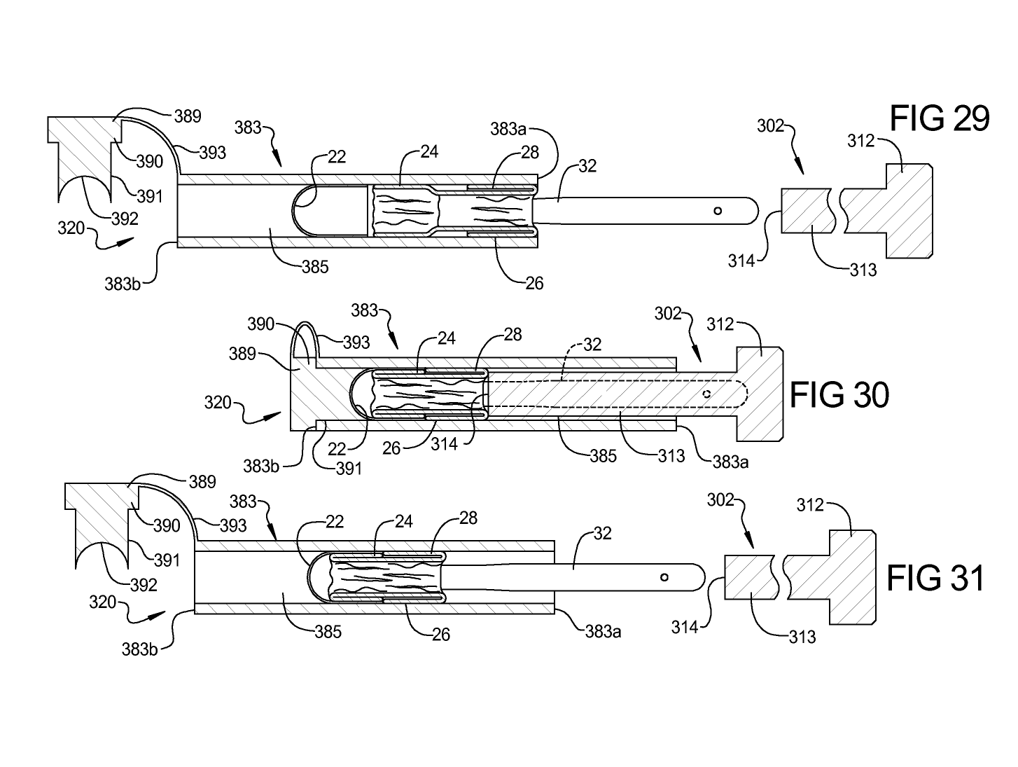

To use loading tool 320, strap 32 of voice prosthesis 26 is inserted into cavity 385 through plug end 383b of barrel 383. Strap and voice prosthesis 26 are pushed into cavity 385 until strap 32 extends out of open end 383a of barrel 383. Strap 32 is then pulled out of barrel 383 through open end 383a such that the rest of voice prosthesis 26 is pulled into cavity 385 through plug end 383b. During this movement, tracheal flange 28 and esophageal flange 24 can fold over and deform as voice prosthesis 26 is forced into cavity 385, as shown in FIG. 29. Voice prosthesis 26 is moved into a position within cavity 385 that allows room for gel cap 22 to also be inserted into cavity 385 through plug end 383b and for plug 389 to be inserted into cavity 385 through plug end 383b, as shown in FIGS. 29 and 30. Gel cap 22 is inserted into cavity 385 with interior 40 facing voice prosthesis 26.

Next, stem 391 of plug 389 is inserted into cavity 385 through plug end 383b. Head 390 limits the distance plug 389 can be inserted into cavity 385. Next, as shown in FIG. 30, stem 313 of plunger 302 is inserted into cavity 385 through open end 383a. Loading tool 320 is now ready to be operated to push esophageal flange 24 into interior 40 of gel cap 22. To accomplish this, head 312 of plunger 302 is pushed toward open end 383a of barrel 383.

The movement of plunger 302 relative to barrel causes free end 314 to push on tracheal flange 28 of voice prosthesis 26 and push esophageal flange 24 into interior 40 of gel cap 22. Stop 392 on plug 389 engages with end 38 of gel cap 22 to limit the movement of gel cap 22 relative to voice prosthesis 26.

With esophageal flange 24 retained within interior 40 of gel cap 22, voice prosthesis 26 and gel cap 22 can then be removed from loading tool 320. This can be accomplished in a number of ways. For example, when a portion of strap 32 is still extending out of open end 383a of barrel 383, plunger 302 can be removed from barrel 383 and strap 32 can then be pulled from open end 383a to pull voice prosthesis 26 and gel cap 22 thereon from loading tool 320. Alternatively, plug 389 can be removed from cavity 385 and plunger 302 further inserted into barrel 383. This relative movement between plunger 302 and barrel 383 can push voice prosthesis 26 and gel cap 22 thereon out of plug end 383b of barrel 383. Thus, voice prosthesis 26 with gel cap 22 thereon can be removed from loading tool 320 in a variety of manners. Voice prosthesis 26 with gel cap 22 thereon can then be utilized to insert voice prosthesis 26 into the trachealesophageal puncture 44 of a user, as shown in FIG. 10 and as described above. Thus, loading tool 320 can be utilized to position an esophageal flange 24 of a voice prosthesis 26 into interior 40 of a gel cap 22.

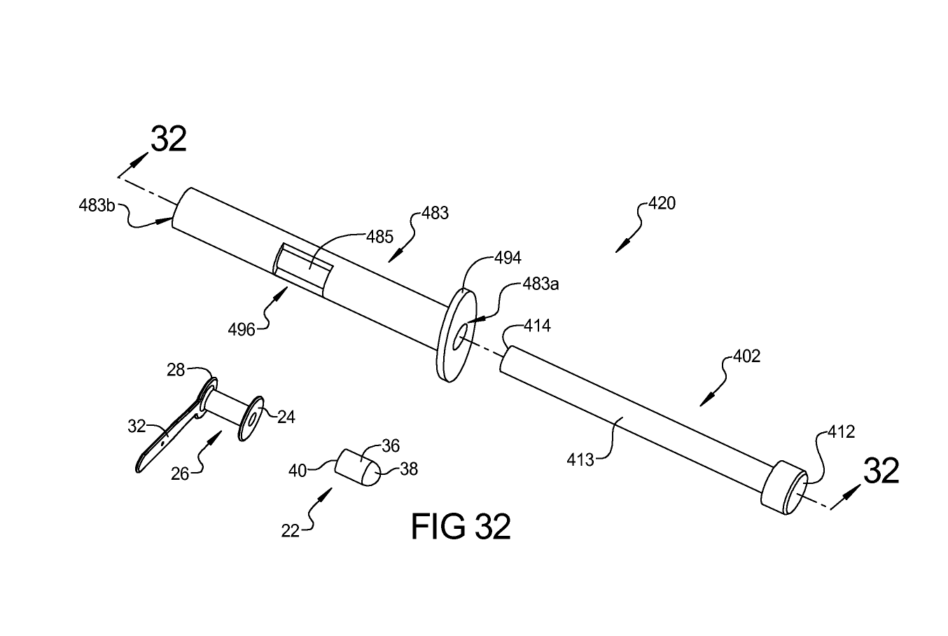

Referring now to FIGS. 32-35, yet another loading tool 420 according to the present disclosure is shown. Loading tool 420 is operable to place gel cap 22 over esophageal flange 24 of voice prosthesis 26. Loading tool 420 includes a barrel 483 and a plunger 402. Barrel 483 is generally cylindrical with an exterior peripheral surface 484 and an interior cylindrical cavity 485 that extends longitudinally throughout the entire length of barrel 483. Cavity 485 is accessible from both ends 483a, 483b of barrel 483. End 483a includes a flange 494 that extends radially outwardly therefrom. Barrel 483 includes an access opening 496 in exterior surface 484. Access opening 496 allows voice prosthesis 26 and gel cap 22 to be inserted into cavity 485, as described below.

Plunger 402 includes a head 412 and an elongated cylindrical stem 413 extending therefrom. Stem 413 has an outer diameter dimensioned to fit within cavity 485 through flange end 483a. Head 412 is larger than the opening to cavity 485 in flange end 483a and limits the travel of plunger 402 within barrel 483. The free end 414 of plunger 402 can include a concave recess to engage with end 38 of gel cap 22. Plunger 402 is operable to push gel cap 22 over esophageal flange 24 of voice prosthesis 26 within barrel 483, as described below.

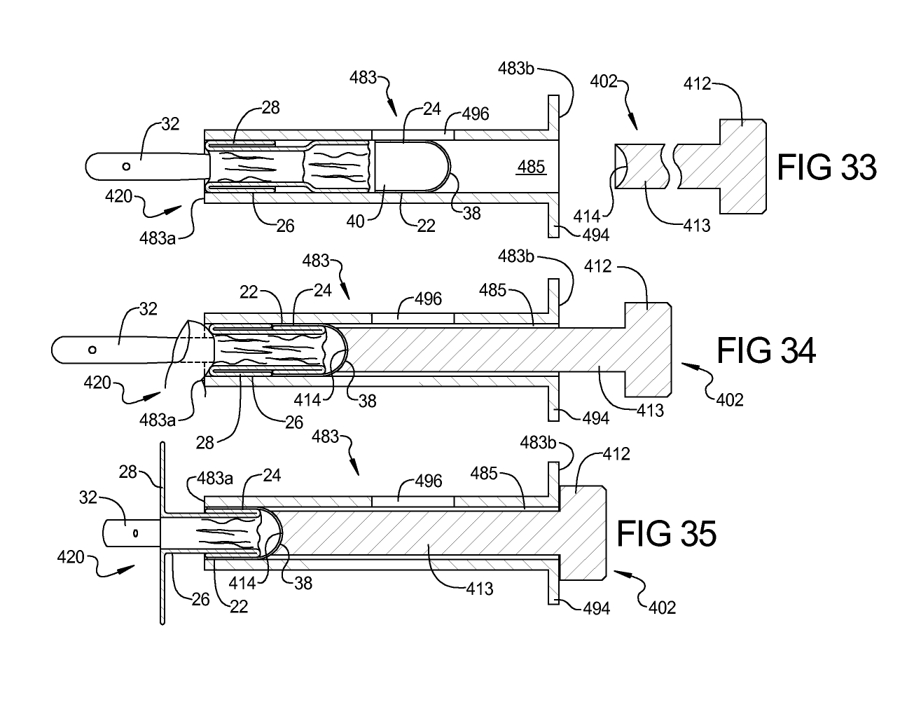

To use loading tool 420, voice prosthesis 26 can be loaded into barrel 483 through access opening 496. Specifically, strap 32 of voice prosthesis 26 is inserted into cavity 485 through access opening 496. Voice prosthesis 26 is pushed through access opening 496 until strap 32 extends out of barrel 483 through end 483b. Next, strap 32 can be pulled to cause the rest of voice prosthesis 26 to enter cavity 485 through access opening 496. As voice prosthesis 26 is pulled into cavity 485, tracheal and esophageal flanges 28, 24 collapse. Voice prosthesis 26 is pulled into cavity 485 until esophageal flange 24 will not interfere with inserting gel cap 22 into cavity 485 through access opening 496. For example, voice prosthesis 26 can be pulled until esophageal flange 24 is clear of access opening 496, as shown in FIGS. 33 and 34.

Next, gel cap 22 is inserted into cavity 485 through access opening 496. Gel cap 22 is oriented such that interior 40 faces voice prosthesis 26 and is open toward end 483b. Next, a finger of the user of loading tool 420 (or other blocking capable member) is placed over end 483b of barrel 483 and plunger 402 is inserted into cavity 485 through flange end 483a, as shown in FIG. 33. Loading tool 420 is now ready to be operated to push gel cap 22 over esophageal flange 24. To accomplish this, head 412 of plunger 402 is pushed toward flange end 483a of barrel 483. This can be accomplished using a syringe-like motion by holding onto flange 494 of barrel 483 while pushing on head 412 of plunger 402.

The movement of plunger 402 relative to barrel 483 causes stem 413 to push gel cap 22 relative to voice prosthesis 26 and onto esophageal flange 24 and a portion of body 30, as shown in FIG. 34. With gel cap 22 fully loaded on voice prosthesis 26, the finger can be removed from end 483b. Voice prosthesis 26 with gel cap 22 thereon can then be removed from unloading tool 420 by continuing to push plunger 402 further into barrel 483 or pulling on strap 32. In both of these manners, voice prosthesis 26 and gel cap 22 thereon can be removed from loading tool 420 through end 483b.

With gel cap 22 and voice prosthesis 26 removed from loading tool 420, voice prosthesis 26 can then be inserted into the trachealesophageal puncture 44 of a user, as shown in FIG. 10 and as described above. Thus, loading tool 420 can be utilized to position a gel cap 22 over an esophageal flange 24 of a voice prosthesis 26.

The various components of loading tools 20, 20′, 120, 220, 320, 420 can be made from a variety of materials. Exemplary materials include polypropylene and ABS plastic. Furthermore, the various components may be transparent or translucent to facilitate the use of the tools. It should be appreciated, however, that other materials can be used and/or one or more of the components may be opaque.

It should be appreciated that while loading tools 20, 20′, 120, 220, 320, 420 are disclosed as being used with a voice prosthesis 26 which is disclosed as being used in a tracheoesophageal puncture 44, loading tools 20, 20′, 120, 220, 320, 420 can be used for other types of medical devices having a radial dimension that is to be reduced by, insertion into a gel cap 22 or other cap for subsequent insertion into an orifice. By way of non-limiting example, loading tools 20, 20′, 120, 220, 320, 420 can be used on a prosthesis intended to be inserted into an opening in a nasal septum or a puncture through a tympanic membrane.

While loading tools 20, 20′, 120, 220, 320, 420 are shown with reference to specific structures and with specific sequences of loading and operating, it should be appreciated that such structures and loading operations are merely exemplary and that deviations to these can occur. For example, the sequence in which voice prosthesis 26, gel cap 22, and the components of loading tool 20, 20′, 120, 220, 320, 420 are assembled together and operated can deviate. As another example, the loading tool can be provided in a double-barrel configuration to accommodate voice prosthesis of varying sizes. That is, multiple barrels can be utilized with each barrel and the associated components dimensioned for different sizes of voice prosthesis such that a single loading tool can be utilized to load gel caps over voice prosthesis of varying sizes. Additionally, other implements can be used in lieu of inserter stick 74. Thus, the loading tools 20, 20′, 120, 220, 320, 420 and their descriptions are merely exemplary and deviations that depart from these exemplary structures and sequence of events are intended to be included in the scope of the present disclosure.

What is claimed is:

1. A loading tool for loading a capsule on a medical device, the loading tool comprising:
a first member having opposite first and second ends and an elongated internal cavity configured to receive a medical device and a capsule therein, at least one of said first and second ends being open and allowing access to said cavity;
a second member having an elongated portion configured to enter said cavity through said at least one open end and engage with at least one of a medical device and a capsule disposed in said cavity,
wherein movement of said second member within said cavity of said first member causes a component of a medical device disposed in said cavity to be inserted into a capsule disposed in said cavity.

2. The loading tool of claim 1, wherein both of said first and second ends are open ends and allow access to said cavity, and the only access to said cavity is through one of said first and second ends.

3. The loading tool of claim 2, further comprising a plug member having a stem and a stop at an end of said stem, said plug member configured to be disposed in one of said first and second ends opposite said second member and wherein said stop engages with and limits movement of one of a medical device and a capsule disposed in said cavity such that movement of said second member within said cavity causes a component of a medical device disposed in said cavity to be inserted into a capsule disposed in said cavity.

4. The loading tool of claim 3, further comprising a connecting member securing said plug member to said first member.

5. The loading tool of claim 3, wherein said stop is concave.

6. The loading tool of claim 1, wherein both of said first and second ends are open ends that allow access to said cavity and further comprising an access opening in an exterior peripheral surface of said first member, said access opening allowing access to said cavity and allowing a medical device and capsule to be inserted into said cavity through said access opening.

7. The loading tool of claim 6, wherein an end of said elongated portion is concave and configured to engage with a convex surface of a capsule.

8. The loading tool of claim 1, wherein the first end is an open end allowing access to said cavity, said second end is a closed end, and further comprising an access opening in an exterior peripheral surface of said first member, said access opening allowing access to said cavity and allowing a medical device and capsule to be inserted into said cavity through said access opening.

9. The loading tool of claim 8, further comprising a cover member operable to cover said access opening in said first member.

10. The loading tool of claim 9, wherein said cover member is an integral member with said first member and is moveable between open and closed positions.

11. The loading tool of claim 8, wherein said cavity has a concave stop adjacent said second end that engages with a convex surface of a capsule disposed therein.

12. A method of inserting a component of a medical device into a capsule, the method comprising:
inserting a medical device into an internal elongated cavity in a first elongated member;
inserting a capsule into said cavity;
inserting an elongated stem of a second member into said cavity through a first end in said first member, said first end being an open end that allows access to said cavity;
directly engaging one of said medical device and said capsule with an end of said stem;
moving at least one of said medical device and said capsule relative to one another in said cavity with said stem of said second member such that a component of said medical device is inserted into said capsule; and
removing said medical device with said capsule thereon from said cavity.

13. The method of claim 12, wherein inserting said medical device includes inserting said medical device into said cavity with a portion of said medical device extending out of said cavity through said first end of said first member.

14. The method of claim 13, wherein removing said medical device with said capsule thereon includes removing said medical device with said capsule thereon through said first end by pulling on said portion of said medical device.

15. The method of claim 12, wherein inserting said medical device and inserting said capsule include inserting said medical device and said capsule into said cavity through a second end of said first member, said second end being an open end allowing access to said cavity and further comprising:
inserting a plug into said second end thereby closing off at least a portion of said second end; and
preventing said medical device and said capsule from being pushed out of said cavity through said second end with said plug during insertion of said component of said medical device into said capsule.

16. The method of claim 12, wherein inserting said medical device and inserting said capsule include inserting said medical device and said capsule into said cavity through an access opening in a peripheral surface of said first member.

17. The method of claim 16, wherein said access opening is the only access opening in said peripheral surface of said first member.

18. The method of claim 16, further comprising closing a cover member thereby covering said access opening with said cover member.

19. The method of claim 12, wherein a second end of said first member opposite said first end is an open end allowing access to said cavity and further comprising blocking said medical device and said capsule from being pushed out of said cavity through said second end during insertion of said component of said medical device into said capsule.

20. The method of claim 12, wherein directly engaging includes directly engaging said capsule with said end of said stem and wherein moving at least one of said medical device and said capsule relative to one another in said cavity with said stem includes pushing said capsule onto said component of said medical device with said stem.
